(19) United States
(12) Patent Application Publication
(10) Pub. No.: US 2021/0235859 A1
(43) Pub. Date: Aug. 5, 2021

(54) DENTAL CARE APPARATUS AND METHOD

(71) Applicant: Nikesh Farmah, Coventry (GB)

(72) Inventor: Nikesh Farmah, Coventry (GB)

(21) Appl. No.: 17/053,935

(22) PCT Filed: May 9, 2019
(86) PCT No.: PCT/GB2019/051274

§ 371 Date: Nov. 9, 2020

(30) Foreign Application Priority Data
May 10, 2018 (GB) 1807630.7

(51) Int. Cl. A46B 15/00; A46B 9/04; A46B 13/02; A61B 1/24; G16H 30/20; G09B 19/00

(52) U.S. Cl. CPC A46B 15/0006; A46B 9/04; A46B 13/02; G09B 19/0084; A46B 15/0038; A61B 1/24; G16H 30/20; A46B 15/0012

(57) ABSTRACT

The invention provides a system for cleaning teeth. The system comprises a smart toothbrush and a smart device. The smart device includes a display. The smart device further comprises a true 3D representation of a user's teeth. The smart toothbrush is arranged to send brushing data to the smart device, and to analyse the brushing data and overlay the brushing data onto the 3D representation of a user's teeth via the display. By providing a true representation of a user's teeth, the system provides a more accurate feedback mechanism to the user, improving the brushing technique of the user.

### CROSS-REFERENCE TO RELATED APPLICATIONS

This is a National Stage Application under 35 U.S.C. § 371 of PCT/GB2019/051274, filed on May 9, 2019, which claims priority to Great Britain Patent Application having serial number 1807630.7, filed on May 10, 2018, which is incorporated by reference herein.

### BACKGROUND OF THE INVENTION

### Field of the Invention

The present invention concerns an apparatus and method for cleaning teeth. More particularly, but not exclusively, this invention concerns an apparatus and method for cleaning teeth that utilises a true representation of the user's teeth to allow for improved brushing technique. The invention also concerns an app which allows the method to be utilised.

### Description of the Related Art

Correct tooth brushing technique may significantly improve dental health, ensuring that all areas of the mouth are correctly and regularly cleaned. Various smart devices currently exist, where brushes including sensors that can detect the position and movement of the brush collect and send that data to a smart device. The smart device collates that data and displays it on a generic representation of a mouth, highlighting any areas that have not been sufficiently cleaned during the tooth brushing process. The present invention seeks to provide an improved system and method for cleaning teeth.

### SUMMARY OF THE INVENTION

The present invention provides a system for cleaning teeth, the system comprising a smart toothbrush; a smart device including a display, the smart device further comprising a 3D representation of a user's teeth; wherein the smart toothbrush is arranged to send brushing data to the smart device, and the smart device is arranged to analyse the brushing data and overlay the brushing data onto the 3D representation of a user's teeth via the display.

Advantageously, the system provides a true representation of a user's teeth with which the brushing data may be compared. By providing a true representation of a user's teeth, the system provides a more accurate feedback mechanism to the user, improving the brushing technique of the user. The true 3D representation of a user's teeth comprises measurement data obtained from and directly related to the user's teeth.

In addition to overlaying the brushing data on the 3D representation of the user's teeth, the smart device may indicate areas that require additional brushing, or more focused attention, for example, to deal with gaps in teeth, gum disease, fillings, crowns, implants etc. Such an arrangement is advantageous over prior art generic representations of teeth, where no consideration is given to the specific user related dental issues that may require specialised brushing attention or techniques.

### BRIEF DESCRIPTION OF THE DRAWINGS

Embodiments of the present invention will now be described by way of example only with reference to the accompanying schematic drawings of which:

FIG. 1 shows a schematic view of a system according to a first embodiment of the invention;

FIG. 2 shows a flow diagram setting out a method of cleaning teeth according to a second and third embodiment of the invention.

### DETAILED DESCRIPTION

The smart toothbrush may comprise a sensor unit. The sensor unit may comprise one or more position and/or motion sensitive sensors. The sensor unit may comprise one or more accelerometers. The sensor unit may comprise one or more pressure sensors arranged to detect the pressure exerted on the user's teeth by the toothbrush. The sensor unit may comprise a sensor arranged to detect odours, for example odours indicative of bad breath. Areas where particular odours are detected may be indicated on the 3D representation of the user's teeth. The sensor unit may comprise an image sensor, for example a camera. The image sensor may be arranged to detect visual indications of tooth health, for example staining of teeth, plaque, decay, and/or bleeding gums. Such brushing data may be indicated on the 3D representation of the user's teeth. The camera may be arranged to obtain measurement data from and directly related to the user's teeth, from which the 3D representation of user's teeth is created. The smart toothbrush may include an alert signal, for example an indicator light, an audible alarm, or a haptic feedback unit, which is activated in the event that the pressure sensor detects an excess of pressure is being exerted by the toothbrush. The smart toothbrush may be arranged to communicate with the smart device via wireless signals. For example, the smart toothbrush and smart device may communicate via Bluetooth <RTM> or any other wireless communication protocol. The smart toothbrush may comprise a wireless communication unit.

The sensor unit and wireless communication unit may be an integral part of the toothbrush. Alternatively, the sensor unit and wireless communication unit may be separable from the remainder of the toothbrush, such that it may be retrofit to one or more different types of toothbrush. Such a retrofit arrangement may be advantageous for users who do not wish to purchase a new toothbrush.

The smart toothbrush may be an electric toothbrush, for example, an ultrasonic electric toothbrush. Alternatively, the smart toothbrush may be a manual toothbrush. The smart toothbrush may comprise interchangeable heads, such as standard brushing heads, floss heads, and/or interdental heads.

The smart toothbrush may comprise a memory unit, the memory unit arranged to store brushing data when the smart toothbrush is unable to send the brushing data to the smart device. The smart toothbrush may be configured to store the brushing data and then send the brushing data to the smart device when paired with the smart device.

The smart device may comprise a camera. The camera may be arranged to monitor the position of the toothbrush as a user undergoes their cleaning regime. The camera may be used to create the 3D representation of the user's teeth. The camera may be arranged to obtain measurement data from and directly related to the user's teeth, from which the 3D representation of user's teeth is created. The smart device may create the 3D representation of the user's teeth using photogrammetry. In an alternative arrangement the 3D representation of the user's teeth may be created using specialised dental scanning software and loaded onto the smart device using any suitable conventional data transfer techniques. The specialised dental scanning software may be arranged to obtain measurement data from and directly related to the user's teeth, from which the 3D representation of the user's teeth is created. The 3D representation of the user's teeth may include additional data, such as indications of special areas of concern as highlighted by a dental professional, or via automated analysis of the 3D representation (for example, highlighting gaps between teeth). The 3D representation of a user's teeth may be created from several scans or images of the user's teeth taken over an extended time period.

According to a second aspect, the invention provides a method of cleaning teeth, the method comprising the following steps: providing a smart toothbrush; providing a smart device, the smart device including a display showing a 3D representation of a user's teeth; a user brushing their teeth with the smart toothbrush and the smart toothbrush sending brushing data to the smart device; the smart device analysing the brushing data and overlaying the data onto the 3D representation of the user's teeth.

The method may comprise the step of creating a true 3D representation of a user's teeth from measurement data obtained from and directly related to the user's teeth.

The method may comprise the additional step of the smart device providing real-time brushing guidance during the teeth cleaning procedure. For example, the smart device may indicate, on the 3D representation of the user's teeth, areas which require additional brushing attention. Such an indication may be via colour changes, arrows, or any other suitable icon. When a child is the user, the indication may comprise cartoon characters or icons to encourage good brushing behaviour. Alternative or additional indications of where particular brushing attention is required may include audible feedback, such as the smart device or smart toothbrush beeping when the smart toothbrush is in a problem area, and/or haptic feedback, such that the smart toothbrush vibrates when in a problem area.

The method may include the additional step of the smart device forming the 3D representation of user's teeth, for example by a camera of the smart device taking a number of pictures or videos of the user's teeth. Alternatively, the method may comprise the step of forming a 3D representation of a user's teeth using specialist dental scanning software, and loading that 3D representation onto the smart device. The 3D representation of the user's teeth may comprise additional data, for example areas of the user's teeth that require specific brushing attention. These areas may be gaps in teeth, gum disease, fillings, crowns, implants etc.

The method may comprise the step of the smart device storing the brushing data of a user and analysing that data in order to determine whether there are any undesirable user-specific brushing habits. For example, the user may regularly miss out properly brushing wisdom teeth because of access issues. If this is the case, the smart device may indicate such areas on the 3D representation of the user's teeth. Alternatively or additionally, the smart device may provide real-time instructions to a user to improve their brushing technique, for example, by improving access to hard to reach areas by increasing or decreasing how widely the mouth is opened. The brushing data stored by the smart device may be sent to third parties, for example, to a user's dentist or insurance provider, or stored for potential use in medical/legal disputes. The brushing data may be used to ensure a treatment regime is being correctly followed, and/or to reduce insurance premiums. The brushing data may be used for evidence in medical/legal disputes, for example between the user and an a dentist. The method may include the step of a user taking regular images of their teeth, and the smart device generating regular 3D models of the user's teeth, which may be beneficial as evidence in the case of any medical/legal disputes arising between the user, a dentist, and/or an insurance company. The regular 3D models of the user's teeth may be compared to show any transitions or changes in health status, such as reduced staining, reduced plaque etc. over time. The regular 3D scans may be shown overlapped or side by side to allow comparison of the scans over time. For example the progression or regression of gum disease or teeth colour over a 3 year period.

The brushing data may be analysed in order to provide incentives to a user for correct teeth brushing. For example, consistently good brushing behaviour may build up rewards. For some users, for example younger users, the brushing data may be used as part of a game, with consistently good brushing behaviour resulting in game related rewards.

The method may also comprise the step of the smart device sending prompts to a user, for example to undertake regular teeth cleaning, and/or to send reminders regarding dental check-ups.

According to a third aspect of the invention, there is provided a computer program comprising a set of instructions, which, when executed, cause a computerised device to perform a method including the following steps: generating and displaying a 3D representation of a user's teeth; receiving data from a smart toothbrush, the data relating to the brushing technique of a user cleaning their teeth; the smart device analysing the brushing data and overlaying the data onto the 3D representation of the user's teeth, and displaying the 3D representation of the user's teeth with the brushing data overlaid.

The computer program may further comprise instructions which cause the computerised device to create a 3D representation of a user's teeth by manipulation of one or more images or measurements of the user's teeth taken by the computerised device. The computerised device may be a smart phone, a tablet device, or any other suitable computerised device as will be appreciated by the skilled person.

It will of course be appreciated that features described in relation to one aspect of the present invention may be incorporated into other aspects of the present invention. For example, the method of the invention may incorporate any of the features described with reference to the apparatus of the invention and vice versa.

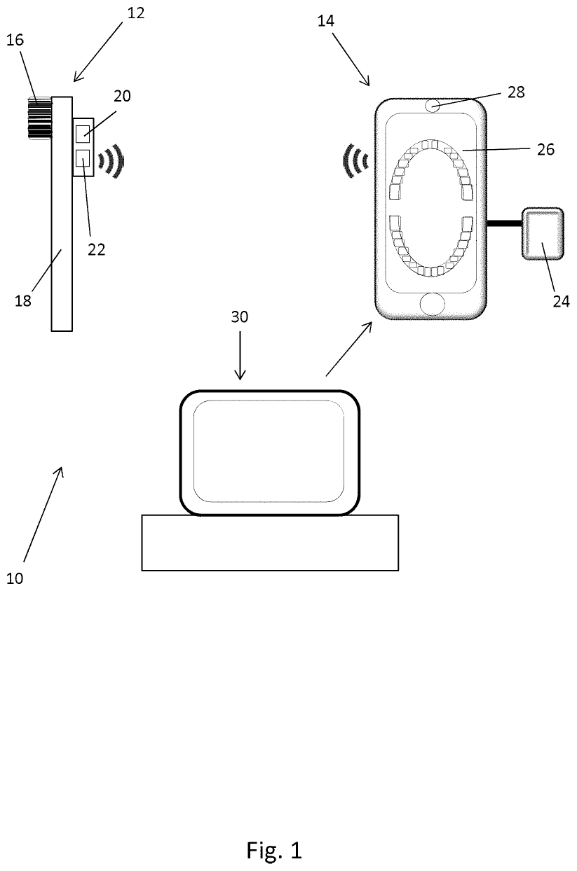

FIG. 1 shows a system 10 which comprises a smart toothbrush 12 and a smart device 14. The smart device may be any suitable smart phone or tablet device, as would be understood by the skilled person. The smart device may be operated by selection of an app, that app including the necessary programming for the smart device to interact with the smart toothbrush 12 and carry out the various steps described below. Delivery of a smart device based system in this way is conventional and would be understood by the skilled person as such. Therefore, the following description addresses the present invention at a relatively high schematic level, as would be clearly understood by the skilled person, and at a level at which the invention could easily be put into effect with routine app developmental work. The smart toothbrush includes a brush head 16 attached to the main body 18 of the smart toothbrush 12. In this case, the toothbrush 12 is electrically operated, and the brush head 16 is removable and replaceable, either with a new or differently configured brush head. In an alternative embodiment, the smart toothbrush may be manually operated, and the brush head 16 may be permanently attached to the main body 18. A sensor unit comprising an accelerometer 20 is provided on the main body 18 of the smart toothbrush, along with a wireless communications unit 22, configured in this case to use the Bluetooth <RTM> communication protocol. The accelerometer is arranged to collect brushing data during use of the smart toothbrush 12, for example relating to the position, angle, and direction of movement of the smart toothbrush 12. The wireless communications unit 22 is arranged to receive the accelerometer 20 data and transmit the data to the smart device 14 for analysis. The accelerometer 20 and the wireless communications unit 22 may be an integral part of the smart toothbrush 12, or they may be retrofit to a “non-smart” toothbrush in order to turn any toothbrush into a smart toothbrush.

The smart device 14 comprises a processor 24 which receives the brushing data from the wireless communications unit 22. The smart device 14 comprises a 3D model of the user's teeth, which may be created by the smart device 14 itself or may be loaded onto the smart device from specialist 3D dental modelling applications 30. In order to create the 3D model using the smart device 14, a camera 28 which forms part of the smart device 14 is used, for example with a photogrammetry technique as will be understood by the person skilled in the art. Specialist 3D dental modelling applications may include any of 3Shape, Cerec, and iTero. The 3D model of the user's teeth may be displayed on a display screen 26 of the smart device 14. During the brushing process, the brushing data collected by the processor 24 may be analysed and overlaid onto to the 3D representation of the user's teeth. The 3D representation of the user's teeth may include additional information including indicators of areas of particular concern or where specialist brushing technique is required, for example where there are gaps in teeth. That information and the display may be reactive to the real time brushing data being collected, for example areas of the representation changing colour when they have been adequately brushed, and/or directional indicators being used to focus the user's attention on a particular area of their teeth. The additional information included as part of the 3D representation may be provided by a dental professional, by the automatic analysis of the 3D model by the smart device 14, or by a user selecting specific areas on the 3D model using an interface with the smart device 14. The brushing data collected by the processor 24 may be stored to provide historic brushing trends, and/or analysed by the processor 24 to identify any habitual brushing errors. The brushing data may also be sent to third parties, for example a dentist or insurance provider, which may allow for directed brushing tips, and/or an adjustment of insurance premiums. The brushing data may also be used as evidence in medical/legal disputes. The user may take regular images and use the smart device to create regular 3D representations of their teeth, and this information may be used as evidence in medical/legal disputes, and/or to provide a dentist with information regarding whether or not a treatment regime is working correctly.

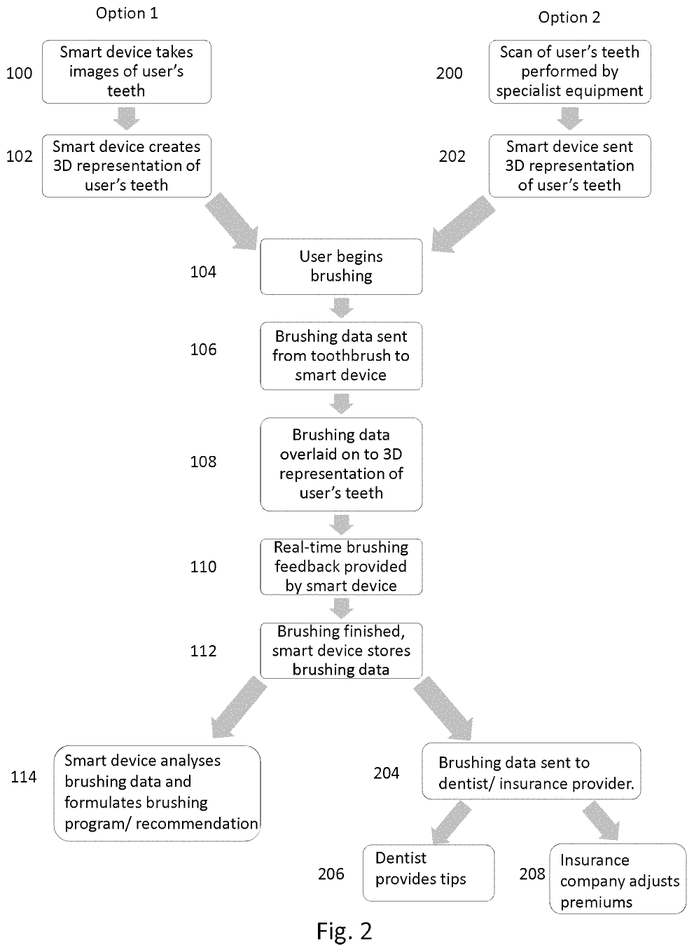

FIG. 2 shows a second and third embodiment of the invention, comprising a method of cleaning teeth using a system as described with reference to FIG. 1. There may be two options available. Option 1 is a user guided process and requires the user to use the smart device 14 to take a picture or pictures of their teeth prior to brushing 100. The smart device 14 may comprise an app (computer program) including the necessary instructions to guide the user through this process. The smart device 14 will then create a 3D representations of the user's teeth using the image taken and the specialist 3D modelling application 102. Such techniques will be understood by the skilled person, and include photogrammetry and other method of creating a three dimensional image. Option 2 is a dental professional led route, and requires a scan of the user's teeth to be performed by a specialist equipment 200 in order to create a 3D model of the user's teeth. This model is then sent to, and uploaded on to the smart device 202. Regardless of the initiation of the process, and in particular how the 3D representation of the teeth is obtained, following this, both options have a common path, with the user brushing their teeth 104. During brushing, the smart toothbrush 12 collects brushing data including properties such as position of the brush, pressure exerted by the brush, and duration of the brushing procedure. The brushing data collected by the smart toothbrush 12 is sent via the wireless communication unit to the smart device 106. The brushing data may be overlaid on the 3D representation of the user's teeth 108. This allows the user to gain real-time brushing feedback provided by the smart device 110. Such real-time feedback may include directions on areas that need additional attention, guidance in brushing technique, and/or some quantitative indication of the quality of brushing, for example an efficiency score. The brushing data may be stored on the smart device 112 for further use. For example, the smart device 14 may analyse the brushing data and formulate a brushing programme or recommendations for the user in the future 114. Such recommendations may be based on trends on the historical brushing data, for example regularly missed areas of the mouth or common brushing mistakes. Alternatively or additionally, the brushing data may be sent to the dentist or an insurance provider 204. The dentist may be able to provide further analysis or tips 206 to the user, delivered via the smart device 14, or the insurance company may be able to adjust insurance premiums 208 which cover the user's dental work.

Whilst the present invention has been described and illustrated with reference to particular embodiments, it will be appreciated by those of ordinary skill in the art that the invention lends itself to many different variations not specifically illustrated herein. By way of example only, certain possible variations will now be described.

The smart toothbrush 12 may comprise various sensors for collecting brushing data, for example image sensors or odour sensors. Those sensors may be arranged to collect data relating to aspects of teeth health, for example teeth staining, plaque coverage, bad breath etc. and indicate the results on the 3D representation of the user's teeth. The smart toothbrush 12 may comprise a memory unit, the memory unit configured to store brushing data when it is not possible to send the brushing data back to the smart device 14. The method may comprise the step of displaying a comparison of 3D representations of the user's teeth over time, for example to show the progression or regression of teeth health data.

Whilst the method as describe above includes the step of real-time feedback being provided to a user, alternatively, the feedback may be provided after completion of the brushing, with a view to improving the brushing technique of the user in the future.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A system for cleaning teeth, the system comprising:
a smart toothbrush;
a smart device including a display, the smart device further comprising a true 3D representation of a user's teeth;
wherein the smart toothbrush is arranged to send brushing data to the smart device, and
the smart device is arranged to analyse the brushing data and overlay the brushing data onto the 3D representation of a user's teeth via the display wherein the true 3D representation of a user's teeth comprises measurement data obtained from and directly related to the user's teeth.

2. (canceled)

3. The system of claim 1, wherein the smart device further comprises a camera, the camera arranged to create the 3D representation of the user's teeth.

4. The system of claim 3, wherein the camera is arranged to obtain measurement data from and directly related to the user's teeth, from which the 3D representation of user's teeth is created.

5. The system of claim 1, wherein the 3D representation of the user's teeth is created using specialised dental scanning software and loaded onto the smart device using any suitable conventional data transfer techniques.

6. The system of claim 5, wherein the specialised dental scanning software is arranged to obtain measurement data from and directly related to the user's teeth, from which the 3D representation of the user's teeth is created.

7. The system of claim 1, wherein the 3D representation of the user's teeth includes additional data.

8. The system of claim 1, wherein in addition to overlaying the brushing data on the 3D representation of the user's teeth, the smart device is configured to indicate areas that require additional brushing, or more focused attention.

9. The system of claim 1, wherein the smart toothbrush comprises a sensor unit.

10. The system of claim 9, wherein the sensor unit comprises one or more position and/or motion sensitive sensors.

11. The system of claim 9, wherein the sensor unit comprises one or more accelerometers.

12. The system of claim 9, wherein the sensor unit comprises one or more pressure sensors arranged to detect the pressure exerted on the user's teeth by the toothbrush.

13. The system of claim 1, wherein the smart toothbrush is arranged to communicate with the smart device via wireless signals.

14. The system of claim 9, wherein the smart toothbrush comprises a wireless communication unit.

15. The system of claim 14, wherein the sensor unit and wireless communication unit are an integral part of the toothbrush.

16. The system of claim 14, wherein the sensor unit and wireless communication unit may be separable from the remainder of the toothbrush.

17. The system of claim 1, wherein the smart toothbrush is an electric toothbrush.

18. The system of claim 1, wherein the smart toothbrush is a manual toothbrush.

19. The system of claim 1, comprising a camera arranged to monitor the position of the toothbrush as a user undergoes their cleaning regime.

20. A method of cleaning teeth, the method comprising the following steps:
creating a true 3D representation of a user's teeth from measurement data obtained from and directly related to the user's teeth;
providing a smart toothbrush;
providing a smart device, the smart device including a display showing the true 3D representation of a user's teeth;
a user brushing their teeth with the smart toothbrush and the smart toothbrush sending brushing data to the smart device;
the smart device analysing the brushing data and overlaying the data onto the true 3D representation of the user's teeth.

21. The method of claim 20, including the additional step of the smart device providing real-time brushing guidance during the teeth cleaning procedure.

22. The method of claim 20, including the additional step of the smart device indicating, on the 3D representation of the user's teeth, areas which require additional brushing attention.

23. The method of claim 20, including the additional step of the smart device obtaining measurement data from and directly related to the user's teeth, thereby forming the true 3D representation of user's teeth.

24. The method of claim 20, including the additional step of obtaining measurement data from and directly related to the user's teeth using specialist dental scanning software thereby forming the true 3D representation of the user's teeth, and loading that 3D representation onto the smart device.

25. The method of claim 20, further comprising the step of the smart device storing the brushing data of a user and analysing that data in order to determine whether there are any undesirable user-specific brushing habits.

26. The method of claim 25, further comprising the step of the smart device indicating such areas where there are undesirable user-specific habits on the 3D representation of the user's teeth.

27. The method of claim 20, comprising the additional step of the smart device providing real-time instructions to a user to improve their brushing technique.

28. A computer program comprising a set of instructions, which, when executed, cause a computerised device to perform a method including the following steps:
generating and displaying a true 3D representation of a user's teeth;
receiving data from a smart toothbrush, the data relating to the brushing technique of a user cleaning their teeth;
the smart device analysing the brushing data and overlaying the data onto the 3D representation of the user's teeth, and displaying the 3D representation of the user's teeth with the brushing data overlaid.
